(19) United States
(12) Patent Application Publication
(10) Pub. No.: US 2026/0245745 A1
(43) Pub. Date: Aug. 20, 2026

(54) METHOD OF OPERATION OF A DRUG LIBRARY ON A SERVER, METHOD OF OPERATION OF AN INFUSION DEVICE, SERVER AND INFUSION DEVICE

(71) Applicant: Fresenius Vial SAS, Brézins (FR)

(72) Inventor: Vincent Chaboud, Doissin (FR)

(21) Appl. No.: 19/160,678

(22) PCT Filed: May 22, 2024
(86) PCT No.: PCT/EP2024/064042

§ 371 Date: Aug. 28, 2025

(30) Foreign Application Priority Data
May 24, 2023 (EP) 23315209

(51) Int. Cl. G16H 70/40; G16H 20/17

(52) U.S. Cl. CPC G16H 70/40; G16H 20/17

(57) ABSTRACT

A method of operation of a drug library on a server, wherein the server comprises a memory unit for storing data. The method comprises the steps of creating at least one profile on the server, wherein creating the at least one profile comprises allocating in the memory unit a total amount of memory size for the at least one profile, and adding a current drug profile of the drug library to the at least one profile. Adding the current drug profile comprises determining a memory requirement of the current drug profile and determining a remaining amount of memory of the at least one profile, wherein the remaining amount of memory of the at least one profile depends on the memory requirement of the current drug profile.

The invention relates to a method of operation of a drug library on a server, a method of operation of an infusion device, a server for operation of a drug library and an infusion device.

Infusion devices, in particular infusion pumps, are used to administer medication, analgesics and anesthetics to a patient. The parameters determining the administration are entered by a clinician into the pump using a pump user interface. In order to support the clinician in defining the parameters for administration, a drug-library profile is usually provided on the infusion device. The profile comprises a number of drug profiles, wherein for each drug profile drug data are stored. Drug data comprise drug names, doses, limits to upper and lower ranges of administration parameters, flowrate limits, programming modes, etc. Different profiles may be defined for different stations or wards on which the infusion device is used. For example, one profile may be defined for an emergency unit and another profile may be defined for an intensive care unit.

Profiles are provided to an infusion device by a server. On the server, the profiles are generated, e.g. by a pharmacist or biomedical engineer, from a drug library. Hence, the profiles are user-configurable.

The profile is e.g. downloaded from a server hosting the drug library to an internal memory unit of an infusion device. Once downloaded to the infusion device, the drug-library profile can then be accessed by a processing unit or processing circuitry of the infusion device. Multiple profiles can be stored on a memory unit of an infusion device.

On the infusion device, as well as on the server, a defined amount of memory is allocated for a profile. This is due to the limited storage space available on an infusion device. As a consequence, presently, the number of drug profiles storable for each profile is limited to a fixed number. This number is obtained from a maximum storage requirement for a drug profile divided by the maximal amount of memory available for drug-data storage on an infusion device. For example, the maximum number of drug profiles in a profile is 200.

Hence, the total memory allocated on an infusion device and the maximum number of drug profiles in a profile are fixed independent of actual usage and requirements. This leads to an inefficient use of memory size available. This is particularly detrimental for the infusion device.

It is an object of the instant invention to provide a method of operation of a drug library on a server as well as a method of operation of an infusion device, which allows a more efficient usage of memory available for drug-data storage on a server, but also and in particular on an infusion device.

This object is achieved by means of a method comprising the steps of claim 1.

Accordingly, the server comprises a memory unit for storing data and the method comprises the steps of creating at least one profile on the server, wherein creating the at least one profile comprises allocating in the memory unit a total amount of memory for the at least one profile and, adding a current drug profile of the drug library to the at least one profile. According to the invention, the step of adding the current drug profile comprises determining a memory requirement of the current drug profile and determining a remaining amount of memory of the at least one profile, wherein the remaining amount of memory of the at least one profile depends on the memory requirement of the current drug profile.

In this way, the memory required for storage of the at least one drug profile is dynamically determined and allocated. It is thus possible to determine how much memory is required for storage of each individual drug profile. Hence, there is no necessity to prescribe a maximum number of drug profiles that can be added to a profile in dependency of a maximum or fixed storage requirement of a drug profile. To the contrary, by determining the remaining amount of memory of the at least one profile, it can be dynamically determined whether a further drug profile can be added to the profile. This allows for a more efficient use of available memory size.

Moreover, as the actual memory requirement of the profile is known from the (actual) memory requirements of added drug profiles, the memory needed for storage of the profile on an infusion device, can be dynamically allocated. Unused memory can be freed and made available to other processes on the server, but in particular also on the infusion device. Considerable memory savings result.

The total amount of memory is, for example, determined by the memory size of the memory unit of an infusion device, e.g. the entire memory size of the memory unit or part of the memory size of the memory unit in case that only part of the memory size is reserved for storage of drug-library data. Alternatively, the total amount of memory is a fixed defined value, e.g. 120 kB. A memory requirement of a current drug profile is the actual memory required for storage of that current drug profile. This is dynamically determined and reflects the actual memory requirement of that drug profile in dependence of its actually added drug data in contrast to a hypothetical maximum memory assigned statically in previous state of the art methods. This allows adding more drug profiles to a profile if the hypothetical maximal memory requirement is not exhausted by at least some of the drug profiles added to the profile, e.g. because they contain less than the maximal amount of drug data.

The method comprises repeating the step of adding a current drug profile of the drug library to the at least one profile. During each addition, the memory requirement of the current drug profile, i.e. of the just to-be added drug profile, is determined as well as the remaining amount of memory of the at least one profile. The method terminates when all drug profiles to be added to the at least one profile have been added or when the total amount of memory is exhausted. This means, that the remaining amount of memory is less than the memory requirement of a further drug profile. This allows to maximize the number of storable drug profiles per profile depending on the memory requirements of the added drug profiles.

The drug profiles are added to the profile e.g. by a pharmacist or by a biomedical engineer. Different profiles can be generated in this way. For example, different profiles for different wards or stations can be generated.

According to one aspect, the remaining amount of memory of the at least one profile depends additionally on the total amount of memory of the at least one profile and a sum of memory requirements of drug profiles previously added to the at least one profile and the memory requirement of the current drug profile. In this way, it is possible to track the remaining amount of memory available for adding further drug profiles to the at least one profile. For example, the remaining amount of memory is the difference between the total amount of memory of the at least one profile and the sum of memory requirements of drug profiles previously added to the at least one profile and the memory requirement of the current drug profile. In particular, a number of drug profiles are added to the profile succinctly. Upon each addition, the remaining amount of memory is determined.

According to one aspect, a maximum number of storable drug profiles is determined dynamically based on the remaining amount of memory. Hence, a maximum number of storable drug profiles is dynamically determined based on actual memory requirements. In contrast to a fixed maximum number of storable drug profiles that is static, the dynamic determination of the maximum number of storable drug profiles allows to store more drug profiles to the at least one profile in case the added drug profiles do not exhaust their maximum storage requirement. This allows for an efficient use of memory, on the server as well as on an infusion device to which the profile has been downloaded. In one aspect, drug profiles are added by a user succinctly, until all drug profiles the user wishes to add have been added or until the remaining amount of memory is exhausted or is below a minimum value. The minimum value is for example determined by a minimal storage requirement of a drug profile. Alternatively, the minimum value is the memory requirement of the drug profile to be added. That means, the server allows for the addition of further drug profiles until the remaining amount of memory is zero or below the minimal value. In this way, the maximum number of storable drug profiles is determined dynamically. The provision of a static, fixed maximum number of storable drug profiles is avoided.

According to a further aspect, the remaining amount of memory is output to a user, for example via a progress bar. In this way, immediate feedback on the remaining available memory for storage of the profile is provided. This allows a user defining the profile to decide how many and which drug profiles, if any, to add to the profile. Additionally, in one example, the output comprises a prompt to confirm an addition of the current drug profile to the profile. Hence, before the current drug profile is added to the profile, a confirmation is needed by a user. This provides an additional check.

According to another aspect, the minimum number of current drug profiles which can be added to the profile is determined based on the remaining amount of memory and a maximum memory requirement of a drug profile. The maximum memory requirement of a drug profile is the maximum amount of memory that a drug profile is allowed to require. This value is, e.g., predefined, for instance as part of the drug library configuration or as part of the server configuration. Alternatively or additionally, this minimum number of current drug profiles is output to a user, e.g. as a number on a display. Correspondingly, in one example, a maximum number of current drug profiles which can be added to the profile is determined based on the remaining amount of memory and a minimum memory requirement of a drug profile. The minimum memory requirement of a drug profile is the minimum amount of memory that a drug profile occupies, i.e. the amount of memory that a drug profile occupies if filled according to its minimum requirements (e.g. only drug name specified). This value is, for instance, predefined. In one example, this maximum amount of current drug profiles is output to a user, e.g. as a number on a display.

In one aspect, adding the current drug profile comprises determining a weight of the current drug profile. The weight of the current drug profile depends on the memory requirement of the current drug profile and a weight classification scheme. The weight classification scheme hence provides a categorization of drug profiles according to their memory requirement. This allows to provide a more detailed determination of the number of drug profiles that can still be added to the profile. For example, the weight classification scheme comprises a plurality of weight classes, wherein each weight class is associated with a memory interval. E.g., the weight classification scheme comprises, for example, three weight classes (“light”, “medium”, “heavy”). The weight class of the current drug profile is determined depending on its memory requirement. E.g. the current drug profile is assigned the weight class associated with the memory interval into which the memory requirement of the current drug profile falls. For example, the current drug profile is classified as “light” if the memory required for storing it is less than 200 bytes. It is classified as “medium” if the memory needed for storing it is more than or equal to 200 bytes and less than 400 bytes. If its memory requirement is equal to or exceeds 400 byte, it is classified as “heavy”. In this way, a more detailed prediction of the number of drug profiles to be stored in the profile can be provided, also to a user. E.g. the output can specify that either two light or one medium drug profile can be added to a profile if the remaining amount of memory is 400 byte. This provides the user with greater flexibility when defining the profile.

In one aspect, the method comprises the step of creating a current drug profile in the drug library, wherein the step of creating a drug profile comprises adding drug data, for example through user input e.g. from a drug library. Drug data comprise drug name, dosage data, administration information, etc. In one example, the step of creating a current drug profile comprises determining a weight of the current drug profile. Hence, the weight of the current drug profile is already determined upon creation. This weight is in one aspect output to a user already upon creating of the drug profile. In one aspect, the weight of the current drug profile is determined repeatedly during creation of the current drug profile, in particular, the weight is determined repeatedly as more and more drug data are added. For example, the weight is determined depending on the current memory requirement of the current drug profile. The current memory requirement refers to the memory that would be required to store the current drug profile at its current state. For example, the current drug profile is created by succinctly adding drug data to the current drug profile. After addition of each drug data, the current memory requirement of the drug profile is determined. This weight is, for instance, regularly output to a user as the user adds drug data to the drug profile. Hence, the user has feedback on the size of the drug profile the user is about to create.

According to one aspect, the method comprises the step of generating a dataset, wherein the dataset comprises the at least one profile. The dataset hence is an individualized drug library, usually generated from a large drug library hosted e.g. on the server. Depending on needs and requirements, one or several profiles are stored in the dataset. In one aspect, the method comprises the step of downloading the dataset to an infusion device, wherein downloading the dataset to the infusion device comprises pushing, by the server, the dataset to the infusion device or pulling, by the infusion device, the dataset from the server. In this way, an individualized drug library is provided on an infusion device, generated according to the needs and requirements associated with that infusion device. Server and infusion device are, e.g., connected via a wired or wireless communication connection. Server and infusion device belong, for example, to a common network such as a LAN or WLAN.

Downloading the dataset to an infusion device comprises in one aspect transmitting dedicated commands to the infusion device. According to one aspect, transmitting all the drug data for a drug profile comprises transmitting a write command (e.g. 1 byte), transmitting the total size of the drug profile (e.g. 1 byte), transmitting a zone to store the drug profile (e.g. 1 byte for external TCI, regular or drug list zone) and transmitting a drug index. Then for each drug data (drug parameter), the total size of that drug data (e.g. 1 byte) and the drug data itself (e.g. drug name, usually multiple bytes) are transmitted.

The object of the present invention is likewise achieved by a method of operation on an infusion device comprising a memory unit for storing data, wherein the method comprises the steps of downloading a dataset from a server, wherein the dataset comprises at least one profile, wherein the at least one profile comprises at least one drug profile and wherein the at least one drug profile comprises drug data. The method further comprises the step of storing the dataset to the infusion device, wherein storing the dataset comprises contiguously storing the at least one drug profile of the at least one profile in the memory unit of the infusion device. In particular, the dataset has been generated on a server according to a method comprising the features of claims 1 to 11. Note that the dataset may comprise only the at least one profile.

In this way, the amount of memory blocked on the infusion device is defined by the actual amount of memory required for storage of the dataset. No unused memory is wasted due to a static allocation of memory on the infusion device depending on a maximum amount of memory required by a dataset or profile. This leaves unused memory available on the infusion device for other tasks. A more efficient use of memory on the infusion device is hence provided. Moreover, the available memory is used more efficient in the sense that the number of drug profiles storable for the at least one profile is determined depending on the actual memory requirement of each drug profile. This allows to maximize the number of drug profiles storable for the at least one profile.

For example, the infusion device receives the dedicated commands and stores the drug data contiguously in a memory unit of the infusion device, e.g. an external flash memory. In one aspect, storing the dataset of the infusion device comprises adding cyclic redundancy checks for the drug data. In this way, the complete and correct transmission of drug data can be monitored.

The object of the present invention is likewise achieved through a server for operation of a drug library, wherein the server comprises processing circuitry. The processing circuitry is configured to execute a method according to any of the claims 1 to 11.

A further subject of the present invention is an infusion device comprising processing circuitry which is configured to execute a method according to any of the claims 12 to 13.

The idea underlying the invention shall subsequently be described in more detail with respect to the embodiment shown in the drawings. Herein:

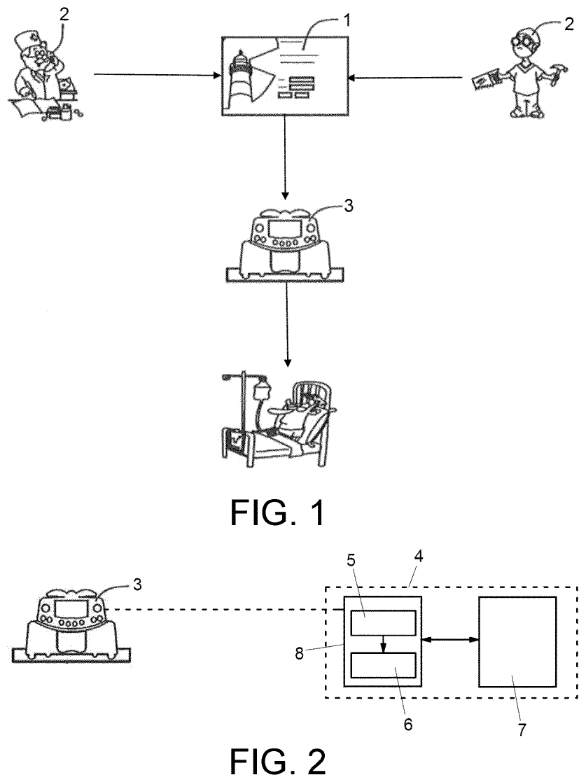

FIG. 1 illustrates a method of operation of a drug library on a server and a server according to an embodiment of the present invention;

FIG. 2 illustrates a method of operation on an infusion device and an infusion device according to an embodiment of the present invention;

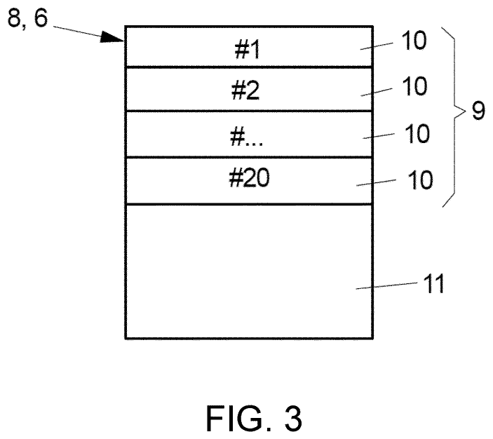

FIG. 3 illustrates a method of operation of a drug library on a server and a method of operation on an infusion device according to an embodiment of the present invention;

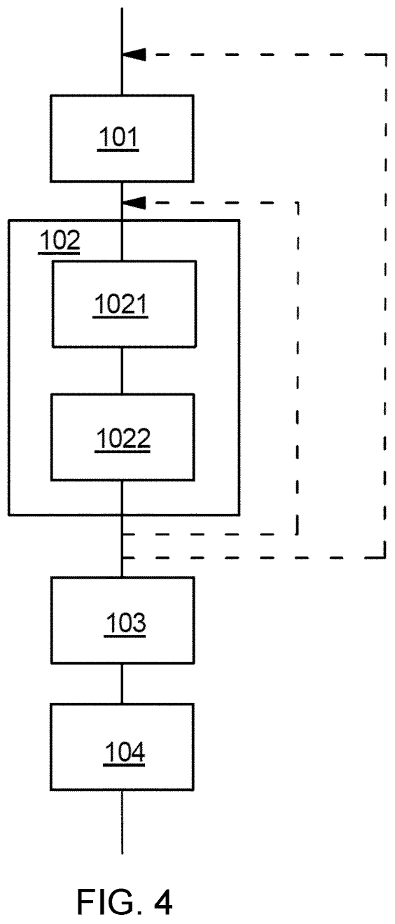

FIG. 4 illustrates a method of operation of a drug library on a server and a method of operation on an infusion device according to the prior art;

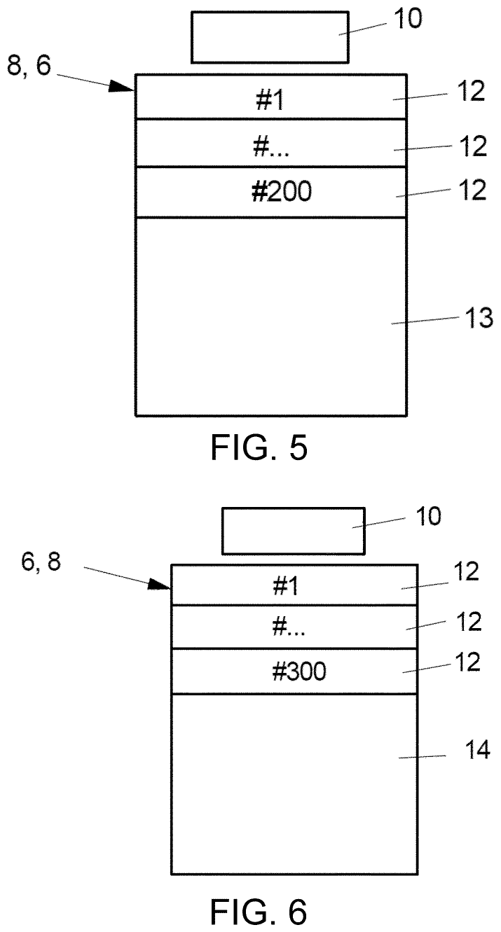

FIG. 5 illustrates a method of operation of a drug library on a server and a server according to an embodiment of the present invention;

FIG. 6 illustrates a method of operation on an infusion device and an infusion device according to an embodiment of the present invention; and

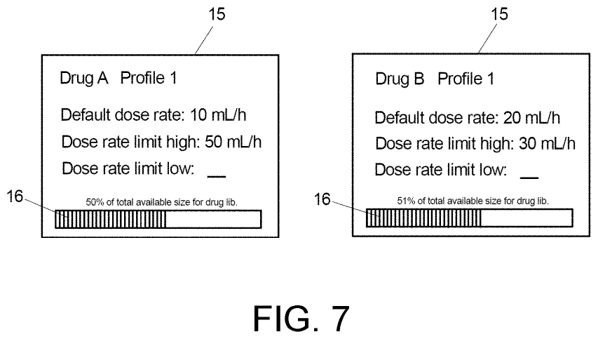

FIG. 7 illustrates a method of operation of a drug library on a server and a method of operation on an infusion device according to an embodiment of the present invention.

FIG. 1 shows a method of operation of a drug library on a server 1 and on an infusion device 3 in an exemplary hospital setting. The server 1 hosts or stores a drug library comprising drug data such as drug names, dosage regimes, etc. Profiles 10 (see FIG. 3) can be generated using the data provided by the drug library which comprise drug data relevant for specific settings. A profile 10 is generated and configured for a specific purpose. For example, different profiles 10 may be provided for different stations or wards of a hospital. A profile 10 is generated on the server 1 by a user 2, such as a pharmacist or biomedical engineer. Each profile 10 comprises one or a multitude of drug profiles 12 (see FIGS. 4, 5) comprising drug data for a specific drug each. Several profiles 10 may be stored in a common dataset. The dataset comprising a single profile 10 or multiple profiles 10 is downloaded to an infusion device 3, providing the infusion device 3 with all relevant drug data needed in its setting. For example, dataset and its profiles 10 are part of a dosage error reduction system (DERS). The DERS comprises control algorithms implemented in the infusion device 3 to prevent errors in the choice and programming of dosages. The necessary information e.g. on admissible dosage regimes is obtained from the profile 10 on the infusion device 3. Once the infusion device 3 is programmed, it can start administering medication, analgesics or anesthetics as programmed.

The infusion device 3 comprises processing circuitry 7 configured to download and store the profile 10 as part of the dataset comprising a single or multiple profiles 10 to the infusion device 3, see FIG. 2. In particular, the infusion device 3 comprises a memory unit 8 for storing data to which the dataset comprising a single or multiple profiles 10 is stored. The memory unit 8 comprises a non-volatile memory 6, e.g. a flash memory, to which the dataset comprising a single or multiple profiles 10 is being stored. Optionally, the memory unit 8 also comprises a temporary, volatile memory 5 such as for example a RAM (random access memory) through which the dataset is stored to the non-volatile memory 6. In one example, the processing circuitry 7 and the memory unit 8 are provided on the same control board 4. The memory unit 8 is in this example an internal memory unit. In an alternative embodiment, the memory unit 8 is an external memory unit. Once stored, the processing circuitry 7 can access the dataset and its profile 10 or profiles 10 in memory 8, in particular in non-volatile memory 6.

As FIG. 3 illustrates, a fixed amount of memory 9 is allocated in the memory unit 8, in particular in the non-volatile memory 6, for the storage of the dataset. This fixed amount of memory 9 is defined to be filled by a maximum number of profiles 10. For example, a maximum number of 20 profiles are allowed to be stored in the fixed amount of memory 9. Hence, a fixed amount of memory 9 is allocated in the memory unit 8, in particular in the non-volatile memory 6, for each profile 10. The free amount of memory 11, for example the difference between the total available memory size of the non-volatile memory unit 6 and the fixed amount of memory 9 reserved for storage of a dataset of profiles 10, is available for other programs and applications run on the infusion device 3. The amount of memory 9 reserved for a dataset is fixed and hence, the free amount of memory 11 available for remaining programs and applications is also fixed.

In prior art methods, a static maximum number of drug profiles 12 per profile 10 is defined, see FIG. 4. This ensures that the fixed amount of memory 9 reserved for each profile 10 is not exceeded when all drug profiles 12 are defined and filled at maximum for a given profile 10. For example, the maximum number of drug profiles 12 storable to a profile 10 is 200. If one or several of the drug profiles 12 are not filled at maximum, the amount of memory taken up by the maximum number of drug profiles 12 is less than the maximum amount of memory allotted to a profile 10. The corresponding memory space 13 remains unused.

FIG. 5 illustrates a method of operation of a drug library on a server 1 which overcomes these deficits of the prior art and allows for a more efficient use of available memory, in particular memory of a memory unit 8 or non-volatile memory 6 of an infusion device 3. The method comprises the steps of creating 101 at least one profile 10 on the server 1. Creating 101 the at least one profile 10 comprises defining a total amount of memory for the at least one profile 10. The total amount of memory is a fixed value, for example set depending on the memory size of the non-volatile memory 6 of the infusion device 3 and a maximum number of profiles 10 allowed to be stored on an infusion device 3. Additionally, the fixed value can depend on the share of the memory size of the non-volatile memory 6 reserved for the storage of drug data, in particular a dataset comprising multiple profiles 10. In this way, it is ensured that the memory 6, 8 on the infusion device 3 is not exceeded when a dataset comprising the created profile or profiles 10 is downloaded to the infusion device 3. The method further comprises the step of adding 102 a current drug profile 12 of the drug library to the at least one profile 10. A current drug profile 12 comprises drug data such as a drug name, dosage regimes, programming instructions, flow rates etc. Adding 102 the current drug profile 12 comprises determining 1021 a memory requirement of the current drug profile 12. The memory requirement is the memory required for storing the current drug profile 12. This is determined dynamically and in dependence of the actual drug data entered or used for that current drug profile 12. Further, adding 102 the current drug profile 12 comprises determining 1022 a remaining amount of memory of the at least one profile 10. This remaining amount of memory depends on the memory requirement of the current drug profile 12. In this way, the remaining amount of memory available for the storage of further drug profiles 12 to the profile 10 is determined dynamically in dependence of the actual memory used by the current drug profile 12. In particular, the remaining amount of memory of the at least one profile 10 depends on the total amount of memory as well as a sum of memory requirements of drug profiles 12 previously added to the at least one profile 10 and the memory requirement of the current drug profile 12. I.e. the remaining amount of memory is for example determined as the difference between the total amount of memory and the amount of memory already occupied by drug profiles 12 previously added and currently being added. In particular, the step 102 of adding a current drug profile 12 is repeated until all drug profiles 12 to be added are added or until the total amount of memory available for the at least one profile 10 is exhausted. That means, no current drug profile 12 can be added if the remaining amount of memory of the at least one profile 10 is below a minimum value or below the memory requirement of the current drug profile 12. The minimum value is, for example, the memory requirement of a drug profile 12 filled at minimum or some other predefined value characterizing the smallest allowable drug profile 12. In this way, the maximum number of drug profiles 12 that can be added to the at least one profile 10 is determined dynamically based on the remaining amount of memory. By allowing further drug profiles 12 to be added as long as the total amount of memory for the at least one profile 10 is not exceeded, memory is used efficiently. The step of adding 102 a current drug profile 12 is repeated until all drug profiles 12 are added as intended by the user 2 or until the remaining amount of memory is exhausted. The drug profile 12 can be added from the drug library. It is also possible, however, that the method comprises the step of creating a drug profile 12 in the drug library. Creating a drug profile 12 comprises adding drug data to a drug profile 12, e.g. from the drug library or through user input. The steps of creating 101 a profile 10 and adding 102 a current drug profile 12 is repeated until all profiles 10 have been created or the maximum number of allowable profiles 10 (e.g. 20) is reached. The method further comprises the step of generating 103 a dataset, wherein the dataset comprises the at least one profile 10. Multiple profiles 10 can be added to the dataset, wherein each profile 10 is generated according to the method presently described. The dataset is then downloaded 104 by an infusion device 3, e.g. through a series of dedicated commands transmitted from the server 1 to the infusion device 3. In this way, a dataset of at least one profile 10 or multiple profiles 10 with a maximum number of drug profiles 12 is generated and provided on the infusion device 3, making optimal use of the memory 6, 8 available on the infusion device 3.

FIG. 6 illustrates a method of operation of an infusion device 3 according to an embodiment of the present invention. Shown is a memory unit 8, in particular a non-volatile memory 6, of the infusion device 3. The method comprises the step of downloading 104 a dataset from a server 1, wherein the dataset comprises at least one profile 10. In the illustrated example, only one profile 10 is comprised in the dataset. The at least one profile 10 comprises at least one drug profile 12 which in turn comprises the drug data. The method further comprises the step of storing the dataset to the infusion device 3, in particular to its memory unit 8, for example a non-volatile memory 6. In the present example, the dataset is stored to the non-volatile memory 6 of the infusion device 3. Storing the dataset comprises contiguously storing the at least one drug profile 12 of the at least one profile 10 in the memory unit 8, here non-volatile memory 6, of the device 3. As the number of storable drug profiles 12 has been determined dynamically depending on the remaining amount of memory on the server 1, more than a fixed number of drug profiles 12 have been added to the profile on the server 1. Hence, the available memory size of the memory unit 8, in particular the non-volatile memory 6, is being efficiently used on the infusion device 3. Optionally, storage space not used, e.g. because the dataset comprises only one profile 10 instead of a maximum number of profiles 10, is freed and can be used by the infusion device 3 for other purposes.

FIG. 7 illustrates a method of operation of a drug library on a server 1. The method comprises the steps of the embodiment described with regard to FIG. 5. Additionally, the method comprises the step of providing an output 15 specifying the remaining memory to a user, for example in the form of a progress bar 16. The left panel shows an exemplary output 15 provided to a user after adding a drug profile 12, here the drug profile 12 of a “drug A” to a profile 10. The progress bar 16 shows the remaining amount of memory available for the addition of further drug profiles 12 to the profile 10 just being created. After addition of a further drug profile 12, here labeled “drug B”, more memory is required, the remaining amount of memory is reduced. This is illustrated by the progress bar 16 moving to the right. The progress bar 16 shows the percentage of the total amount of memory available for the profile 10 that is already occupied. Additionally, the output 15 comprises, for example, a prompt to confirm the addition of the current drug profile 12 to the profile 10.

### List of Reference Numerals

1 Server
2 User
3 Infusion device
4 Control board
5 volatile memory
6 non-volatile memory
7 processing circuitry
8 memory unit
9 memory available for drug-data storage
10 profile
11 memory for other use
12 drug profile
13 unused memory
14 memory for storage of further drug profiles
15 output
16 progress bar
101 creating profile on server
102 adding current drug profile to profile
1021 determining memory requirement of current drug profile
1022 determining remaining amount of memory
103 generating dataset
104 downloading dataset to infusion device

1. A method of operation of a drug library on a server, wherein the server comprises a memory unit for storing data, comprising the steps of:
creating at least one profile on the server, wherein creating the at least one profile comprises allocating in the memory unit a total amount of memory for the at least one profile and
adding a current drug profile of the drug library to the at least one profile
wherein the adding the current drug profile comprises determining a memory requirement of the current drug profile and determining a remaining amount of memory of the at least one profile wherein the remaining amount of memory of the at least one profile depends on the memory requirement of the current drug profile.

2. The method according to claim 1, wherein the remaining amount of memory of the at least one profile depends additionally on the total amount of memory for the at least one profile and a sum of memory requirements of drug profiles previously added to the at least one profile and the memory requirement of the current drug profile.

3. The method according to claim 1, wherein a maximum number of storable drug profiles is determined dynamically based on the remaining amount of memory.

4. The method according to claim 1, wherein the remaining amount of memory is output to a user via a progress bar

5. The method according to claim 4, wherein the output comprises a prompt to confirm an addition of the current drug profile to the at least one profile.

6. The method according to claim 1, wherein the adding the current drug profile comprises determining a weight of the current drug profile wherein the weight of the current drug profile depends on the memory requirement of the current drug profile and a weight classification scheme.

7. The method according to claim 6, wherein the weight classification scheme comprises a number of weights, wherein each weight is associated with a memory interval and the current drug profile is assigned the weight associated with the memory interval into which the memory requirement of the current drug profile falls.

8. The method according to claim 1, further comprising the step of creating a current drug profile in the drug library, wherein the step of creating the current drug profile comprises receiving drug data through user input.

9. The method according to claim 1, further comprising the step of generating a dataset, wherein the dataset comprises the at least one profile.

10. The method according to claim 9, further comprising the step of uploading the dataset to an infusion device, wherein the uploading the dataset to the infusion device comprises pushing, by the server, the dataset to the infusion device or pulling, by the infusion device, the dataset from the server.

11. The method according to claim 10, wherein the uploading the dataset to the infusion device comprises transmitting dedicated commands to the infusion device.

12. A method of operation on an infusion device comprising a memory unit for storing data, wherein the method comprises the steps of:
downloading a dataset from a server, wherein the dataset comprises at least one profile, wherein the at least one profile comprises at least one drug profile, wherein the at least one drug profile comprises drug data;
storing the dataset to the infusion device
wherein the storing the dataset to the infusion device comprises contiguously storing the at least one drug profile of the at least one profile in the memory unit of the infusion device.

13. The method according to claim 12, wherein the storing the profile to the infusion device comprises adding cyclic redundancy checks for drug data.

14. A server for operation of a drug library, wherein the server comprises processing circuitry configured to execute the method according to claim 1.

15. An infusion device wherein the infusion device comprises processing circuitry configured to execute the method according to claim 12.
